(19) United States
(12) Patent Application Publication
(10) Pub. No.: US 2024/0345192 A1
(43) Pub. Date: Oct. 17, 2024

(54) COUNTER ROTATING CURRENT COIL FOR MAGNETIC RESONANCE IMAGING

(71) Applicant: Hyperfine Operations, Inc., Guilford, CT (US)

(72) Inventor: Eddy B. Boskamp, Shelton, CT (US)

(73) Assignee: Hyperfine Operations, Inc., Guilford, CT (US)

(21) Appl. No.: 18/616,810

(22) Filed: Mar. 26, 2024

Related U.S. Application Data
(63) Continuation of application No. PCT/US2022/044880, filed on Sep. 27, 2022.
(60) Provisional application No. 63/248,922, filed on Sep. 27, 2021.

(51) Int. Cl. G01R 33/422; G01R 33/34; G01R 33/36

(52) U.S. Cl. CPC G01R 33/422; G01R 33/34046; G01R 33/3642

(57) ABSTRACT

There is provided a radio frequency (RF) coil apparatus for facilitating magnetic resonance imaging (MRI) of at least a part of a patient's body that is positioned within an imaging region of an MRI system. The RF coil apparatus comprises at least one primary RF coil configured to emit RF pulses and generate a first magnetic field during operation of the MRI system and at least one secondary RF coil configured to generate a second magnetic field that, during operation of the MRI system, at least partially counteracts the first magnetic field in an external region outside of the imaging region of the MRI system.

### CROSS-REFERENCE TO RELATED PATENT APPLICATIONS

This application claims the benefit of priority under 35 U.S.C. § 120 as a continuation of PCT Patent Application No. PCT/US2022/044880, filed on Sep. 27, 2022, which claims the benefit under 35 U.S.C. § 119(e) of U.S. provisional patent application Ser. No. 63/248,922 entitled “COUNTER ROTATING CURRENT COIL FOR MAGNETIC RESONANCE IMAGING” filed Sep. 27, 2021, the disclosures of which are incorporated by reference in their entirety herein.

### BACKGROUND

Magnetic resonance imaging (MRI) provides an important imaging modality for numerous applications and is widely utilized in clinical and research settings to produce images of the inside of the human body. MRI is based on detecting magnetic resonance (MR) signals, which are electromagnetic waves emitted by atoms in response to state changes resulting from applied electromagnetic fields. For example, nuclear magnetic resonance (NMR) techniques involve detecting MR signals emitted from the nuclei of excited atoms upon the re-alignment or relaxation of the nuclear spin of atoms in an object being imaged (e.g., atoms in the tissue of the human body). Detected MR signals may be processed to produce images, which in the context of medical applications, allows for the investigation of internal structures and/or biological processes within the body for diagnostic, prognostic, therapeutic, and/or research purposes.

### SUMMARY OF THE INVENTION

A radio frequency (RF) coil apparatus for facilitating magnetic resonance imaging (MRI) of at least a part of a patient's body that is positioned within an imaging region of an MRI system, the RF coil apparatus comprising: at least one primary RF coil configured to emit RF pulses and generate a first magnetic field during operation of the MRI system; and at least one secondary RF coil configured to generate a second magnetic field that, during operation of the MRI system, at least partially counteracts the first magnetic field in an external region outside of the imaging region of the MRI system.

A magnetic resonance imaging system for imaging at least a part of a patient's body that is positioned within an imaging region of the MRI system, the MRI system comprising: a B₀ magnet that produces a B0 magnetic field; and a radio frequency coil apparatus comprising: at least one primary RF coil configured to emit RF pulses and generate a first RF magnetic field during operation of the MRI system; and at least one secondary RF coil configured to generate a second RF magnetic field that, during operation of the MRI system, at least partially counteracts the first magnetic field an external region outside of the imaging region of the MRI system.

A method of operating a radio frequency coil apparatus for facilitating magnetic resonance imaging of at least a part of a patient's body that is positioned within an imaging region of the MRI system, the method comprising: driving at least one primary RF coil with a first current such that the primary RF coil emits RF pulses and generates a first magnetic field during operation of the MRI system; and driving at least one secondary RF coil with a second current such that the secondary RF coil generates a second magnetic field that at least partially counteracts the first magnetic field in an external region outside of the imaging region of the MRI system.

### BRIEF DESCRIPTION OF THE DRAWINGS

Various aspects and embodiments of the disclosed technology will be described with reference to the following figures. It should be appreciated that the figures are not necessarily drawn to scale. For purposes of clarity, not every component may be labeled in every drawing.

FIG. 1 illustrates example components of an MRI system, in accordance with some embodiments of the technology described herein.

FIG. 2A illustrates an example portable MRI system, in accordance with some embodiments of the technology described herein.

FIG. 2B illustrates an example bi-planar B0 magnet configuration and radio frequency (RF) coil configuration of the example MRI system of FIG. 2A, in accordance with some embodiments of the technology described herein.

FIG. 3 illustrates an example RF coil apparatus, in accordance with some embodiments of the technology described herein.

FIG. 4 illustrates an example graph of a stray field generated by the example RF coil apparatus of FIG. 3, in accordance with some embodiments of the technology described herein.

FIG. 5A illustrates an example of an RF coil apparatus comprising a counter rotating current (CRC) coil, in accordance with some embodiments of the technology described herein.

FIG. 5B illustrates an example method of operating an RF coil apparatus comprising a CRC coil, in accordance with some embodiments of the technology described herein.

FIG. 6A illustrates another example RF coil apparatus comprising a CRC coil, in accordance with some embodiments of the technology described herein.

FIGS. 6B-6C illustrate example graphs of the stray field generated by the example RF coil apparatus of FIG. 6A, in accordance with some embodiments of the technology described herein.

FIG. 7 illustrates another RF coil apparatus comprising a CRC coil, in accordance with some embodiments of the technology described herein.

FIG. 8 illustrates another example RF coil apparatus comprising a CRC coil, in accordance with some embodiments of the technology described herein.

FIG. 9A illustrates another example RF coil apparatus comprising a CRC coil, in accordance with some embodiments of the technology described herein.

FIGS. 9B-9C illustrates example graphs of the stray field generated by the example RF coil apparatus of FIG. 9A, in accordance with some embodiments of the technology described herein.

FIG. 10 illustrates a graph comparing the relative stray field reduction with and without use of a CRC coil, in accordance with some embodiments of the technology described herein.

FIG. 11 illustrates a graph illustrating the stray field reduction when a CRC coil is used with an RF coil, in accordance with some embodiments of the technology described herein.

### DETAILED DESCRIPTION

### Introduction

Aspects of the technology described herein relate to systems and techniques for reducing the strength of an effective magnetic field in an external region outside of an imaging region of an MRI system. Some embodiments provide for a radio frequency (RF) coil apparatus comprising one or more primary RF coils that generate a primary magnetic field in the imaging region of the MRI system. The RF coil apparatus further comprises a secondary RF coil configured to generate a secondary magnetic field that at least partially counteracts the primary magnetic field in the external region outside the imaging region. As a result, the strength of the effective magnetic field in the external region is reduced.

Magnetic resonance imaging involves placing a subject to be imaged (e.g., all or a portion of a patient) in a static, homogenous magnetic field B₀ to align a subject's atomic net magnetization (often represented by a net magnetization vector) in the direction of the B₀ field. One or more RF transmit coils are then used to generate a pulsed magnetic field B₁ having a frequency related to the rate of precession of atomic spins of the atoms in the magnetic field B₀ to cause the net magnetization of the atoms to develop a component in a direction transverse to the direction of the B₀ field. After the B₁ field is turned off, the transverse component of the net magnetization vector precesses, its magnitude decaying over time until the net magnetization realigns with the direction of the B₀ field. This process produces MR signals that can be detected by voltages induced in one or more RF receive coils of the MRI system.

In addition, MRI involves using gradient coils to induce gradients in the main magnetic field B₀ so that the MR signal emanating from particular spatial locations within the subject may be identified (e.g., gradient coils are used to spatially encode detected MR signals). An MR image is formed in part by pulsing the RF transmit coil(s) and/or the gradient coils in a particular sequence, referred to as a “pulse sequence,” and using the RF receive coil(s) to sense MR signals induced by the pulse sequence. The detected MR signals may then be processed (e.g., “reconstructed”) to form an image. A pulse sequence generally describes the order and timing in which RF transmit/receive coils and gradient coils operate to prepare the magnetization of the subject and acquire resulting MR data. For example, a pulse sequence may indicate an order of transmit pulses, gradient pulses, and acquisition times during which the receive coils acquire MR data.

The inventors have recognized that the B₁ field generated by RF coils of an MRI system may extend beyond the imaging region of the MRI system. Specifically, a portion of the B₁ field may comprise a “stray field” in an external region outside the imaging region of the MRI system. The stray field does not facilitate imaging by the MRI system, but can have deleterious effects, especially where the MRI system comprises a portable MRI system that may be transported and operated in the vicinity of one of more other electronic devices.

For example, in some medical facilities such as emergency rooms or intensive care units, a portable low-field MRI system may be in close proximity to one or more other electronic devices, including one or more other portable low-field MRI systems or other medical devices, whose performance may impacted by the strength of the stray field generated by the MRI system. For example, when an RF coil of a first MRI system generates RF pulses, the RF pulses may be detected by the coils of a second MRI system located near or within a threshold distance of the first MRI system, particularly in a case where the second MRI system operates using the same Larmor frequency as the first MRI system. This will have a negative impact on the second MRI system; the detected RF pulses will effectively be noise and lower the signal-to-noise ratio (SNR) of the second MRI system thereby degrading the quality of the resulting MRI images. The negative impact of the operation of one MRI system on the other is exacerbated when the MRI systems are closer to each other (e.g., within 15 meters, within 10 meters, within 5 meters, etc.) and/or oriented such that an unshielded portion (e.g., a patient opening) of one MRI system is facing the other (and even worse when unshielded portions of both MRI systems are facing one another).

Indeed, for portable low-field MRI systems, there is a greater chance that multiple MRI systems will operate in close proximity to one another. In addition, there is a greater chance that the portable MRI system will operate in unshielded locations, such as an emergency room, an intensive care unit or ambulance, which does not have built-in features for reducing the strength of the stray field generated by the portable MRI system.

In addition to the deleterious effects of the stray field interfering with nearby electronic devices, regulations may require the stray field strength to be less than a certain level. For example, the International Standard for electromagnetic emission from the Industrial, Scientific and Medical Equipment (CISPR 11, group 2, class A) requires the stray field of an MRI system operating in the 2.7-2.75 MHz frequency range to be less than 43.5 dB A/m.

Therefore, the inventors have recognized that there is a need for active reduction and/or cancellation of the stray field generated by a primary RF coil(s) of an MRI system, and in particular, a portable MRI system. Accordingly, the inventors have developed an RF coil apparatus which counteracts the stray field generated by the primary RF coil(s). Specifically, the RF coil apparatus comprises, in addition to the primary RF coil(s), at least one secondary RF coil to actively reduce and/or cancel the field generated by the primary RF coil(s) in the external region outside the imaging region of the MRI system. That is, the effective magnetic field which results from the superposition of the first magnetic field generated by the primary RF coil(s) and the second magnetic field generated by the at least one secondary RF coil is reduced and/or eliminated by operation of the at least one secondary RF coil.

In some embodiments, the secondary RF coil and the primary RF coil are implemented as a single coil. For example, the secondary RF coil may comprise one or more loops (e.g., one or more conductors) connected in series with the primary RF coil. The one or more loops connected in series with the primary RF coil generate the second magnetic field that counteracts the first magnetic field generated by the primary RF coil in the external region outside of the imaging region. In such embodiments, the secondary RF coil and the primary RF may be electrically connected. For example, where the secondary RF coil and the primary RF coil consist of a single coil, the secondary RF coil may share a drive circuit with the primary RF coil. Therefore, it should be understood that the secondary RF coil described herein is not limited to embodiments where the secondary RF coil is separate from the primary RF coil and that, in some embodiments, the primary RF coil and the secondary RF coil may together form a single coil.

In other embodiments, the secondary RF coil may comprise an RF coil that is separate from the primary RF coil. For example, the secondary RF coil may comprise one or more conductors that are not electrically connected with conductor(s) of the primary RF coil. The primary RF coil and the secondary RF coil may be coupled to separate drive circuits.

In some embodiments, the RF coil apparatus described herein is capable of reducing the stray field by up to approximately 55 dB. In some embodiments, the RF coil apparatus described herein reduces the stray field by at least 50 dB, at least 45 dB, at least 40 dB, at least 35 dB, at least 30 dB, at least 25 db, or at least 10 db. The RF coil apparatus is capable of providing a consistent reduction of the stray field of at least 40 dB at a distance three or more meters away from the imaging region of the MRI system.

In some embodiments, the current in the at least one secondary RF coil may be used to counteract the stray field generated by the primary RF coil(s). In some embodiments, the current in the at least one secondary RF coil may be 180 degrees out of phase with the current in the primary RF coil(s). For example, if the current in the primary RF coil(s) runs clockwise, the current in the at least one secondary RF coil may run counterclockwise. Accordingly, the at least one secondary RF coil may be referred to as a counter rotating current (CRC) coil or coils.

In some embodiments, the stray field may be oriented along multiple axes. Accordingly, the stray field may be considered as having multiple components (e.g., an x-axis component, a y-axis component, and/or a z-axis component). In such embodiments, the at least one secondary RF coil may be configured to counteract one or more components (e.g., each component) of the stray field by generating a secondary magnetic field, which reduces the strength of the effective magnetic field in the external region outside the imaging region along the one or more axes along which the stray field is oriented.

In some embodiments, the at least one secondary RF coil may be a single coil. The single coil may counteract a stray field oriented along one or more axes (e.g., when the at least one secondary RF coil is tilted relative to the x- and y-axes, the at least one secondary RF coil generates a second magnetic field having components oriented along both of the x- and y-axes). In some embodiments, the at least one secondary RF coil comprises multiple coils. Each coil may be oriented along one of the x- or y-axes. In some embodiments, a current in each of the multiple coils may be controlled independently.

Thus, aspects of the technology described herein relate to a radio frequency apparatus comprising a secondary RF coil configured to counteract at least a portion of a magnetic field generated by a primary RF coil. Some embodiments provide for a radio frequency coil apparatus for facilitating magnetic resonance imaging (MRI) of at least a part of a patient's body that is positioned within the imaging region of an MRI system, the RF coil apparatus comprising: at least one primary RF coil configured to emit RF pulses and generate a first magnetic field during operation of the MRI system; and at least one secondary RF coil configured to generate a second magnetic field that, during operation of the MRI system, at least partially counteracts the first magnetic field in an external region outside of the imaging region of the MRI system.

In some embodiments, the first magnetic field extends over at least a portion of the imaging region and the external region outside of the imaging region. The current in the at least one secondary RF coil may be 180 degrees out of phase with the current in the at least one primary RF coil. Therefore, operation of the at least one secondary RF coil may at least partially reduce the strength of an effective magnetic field in the external region outside of the imaging region of the MRI system.

In some embodiments, the at least one RF coil includes only a single conductor. The single conductor may be connected in series with the at least one primary RF coil. In some embodiments, the at least one RF coil comprises multiple conductors. The multiple conductors may be connected in series with the at least one primary RF coil.

In some embodiments, the second magnetic field generated by the at least one secondary RF coil is directed substantially along a single axis. For example, components of the second magnetic field may be generated so as not to deviate more than 5 degrees in direction from a single axis central to the directions of the components of the second magnetic field. In some embodiments, the second magnetic field generated by the at least one secondary RF coil comprises a first component along a first axis and a second component along a second axis substantially perpendicular to the first axis. The at least one secondary RF coil may be tilted related to a third axis substantially perpendicular to the first and second axes.

In some embodiments, the at least one secondary RF coil comprises multiple coils including a first coil and a second coil. The second magnetic field generated by the at least one secondary RF coil comprises a first component along a first axis generated by the first coil and a second component along a second axis substantially perpendicular to the first axis generated by the second coil. The current in the first coil may be generated by a first power source and the current in the second coil may be generated by a second power source different from the first power source.

In some embodiments, the current in the at least one primary RF coil is generated by a first power source and a current in the at least one secondary RF coil is generated by a second power source different than the first power source.

In some embodiments, the RF coil apparatus comprises a frame comprising a first plate and a second plate disposed opposite the first plate, wherein the at least one primary RF coil is wound around the frame in a plurality of turns (e.g., at least 6 turns). In some embodiments, the at least one primary RF coil comprises a plurality of conductors connected in series, the plurality of conductors being wound around the frame and forming the plurality of turns.

In some embodiments, the RF coil apparatus is coupled to a base having a conveyance mechanism for transporting the RF coil apparatus to different locations.

In some embodiments, the at least one primary RF coil and the at least one secondary RF coil may be part of a single RF coil.

In some embodiments, the at least one primary RF coil and the at least one secondary RF coil may be separate coils.

In some embodiments, the at least one primary RF coil and the at least one secondary RF coil are coupled to a common drive circuit, and wherein the at least one secondary RF coil comprises a plurality of counter rotating current loops connected in series with the at least one primary RF coil. In some embodiments, the at least one primary RF coil and the at least one secondary RF coil are electrically coupled. In some embodiments, the at least one primary RF coil and the at least one secondary RF coil are coupled to different drive circuits. In some embodiments, the at least one primary RF coil and the at least one secondary RF coil are not electrically coupled.

In some embodiments, there is provided a magnetic resonance imaging system for imaging at least a part of a patient's body that is positioned within an imaging region of the MRI system, the MRI system comprising: a B₀ magnet that produces a B₀ magnetic field; and a radio frequency coil apparatus comprising: at least one primary RF coil configured to emit RF pulses and generate a first RF magnetic field during operation of the MRI system; and at least one secondary RF coil configured to generate a second RF magnetic field that, during operation of the MRI system, at least partially counteracts the first magnetic field an external region outside of the imaging region of the MRI system.

In some embodiments, the MRI system further comprises an RF shield at least partially surrounding an imaging region of the MRI system, wherein the shield comprises at least one opening for receiving an anatomy of the patient. In some embodiments, the at least one secondary RF coil is disposed inside the RF shield. In some embodiments, the at least one secondary RF coil is disposed at least partially outside of the RF shield. In some embodiments, the at least one secondary RF coil is disposed outside of the RF shield.

In some embodiments, the at least one secondary RF coil is disposed along the at least one opening of the RF shield. In some embodiments, the at least one secondary RF coil comprises a loop having an area larger than an area of the at least one opening.

In some embodiments, the B0 magnetic field produced by the B₀ magnet is less than or equal to 0.2 T and greater than or equal to 0.1 T. In some embodiments, the B₀ magnetic field produced by the B₀ magnet is less than or equal to 0.1 T and greater than or equal to 50 mT.

In some embodiments, there is provided a method of operating a radio frequency coil apparatus for facilitating magnetic resonance imaging of at least a part of a patient's body that is positioned within an imaging region of the MRI system, the method comprising: driving at least one primary RF coil with a first current such that the primary RF coil emits RF pulses and generates a first magnetic field during operation of the MRI system; and driving at least one secondary RF coil with a second current such that the secondary RF coil generates a second magnetic field that at least partially counteracts the first magnetic field in an external region outside of the imaging region of the MRI system. In some embodiments, the second current is 180 degrees out of phase with the first current.

The aspects and embodiments described above, as well as additional aspects and embodiments, are described further below. These aspects and/or embodiments may be used individually, all together, or in any combination, as the technology is not limited in this respect.

As used herein, “high-field” refers generally to MRI systems presently in use in a clinical setting and, more particularly, to MRI systems operating with a main magnetic field (i.e., a B₀ field) at or above 1.5 T, though clinical systems operating between 0.5 T and 1.5 T are often also characterized as “high-field.” Field strengths between 0.2 T and 0.5 T have been characterized as “mid-field” and, as field strengths in the high-field regime have continued to increase, field strengths in the range between 0.5 T and 1 T have also been characterized as mid-field. By contrast, “low-field” refers generally to MRI systems operating with a B0 field of less than or equal to 0.2 T, though systems having a B₀ field of between 0.2 T and 0.3 T have sometimes been characterized as low-field as a consequence of increased field strengths at the high end of the high-field regime. Within the low-field regime, low-field MRI systems operating with a B₀ field of less than 0.1 T are referred to herein as “very low-field” and low-field MRI systems operating with a B₀ field of less than 10 mT are referred to herein as “ultra-low field.”

### Example Magnetic Resonance Imaging Systems

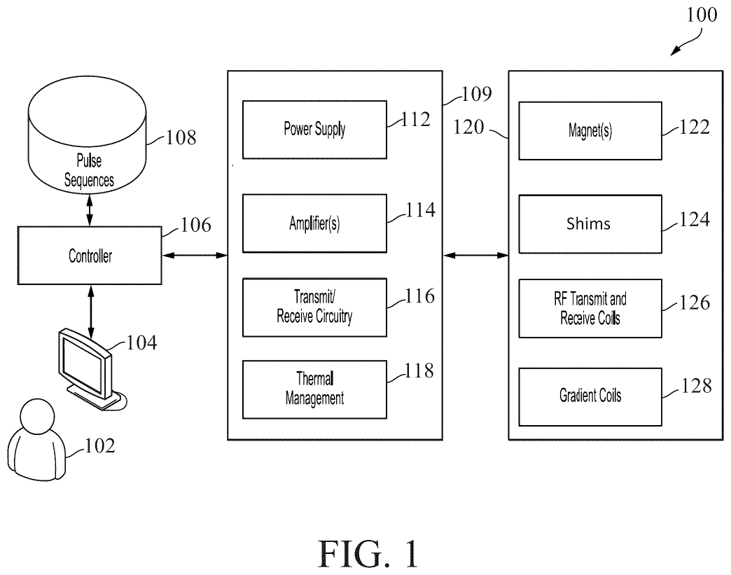

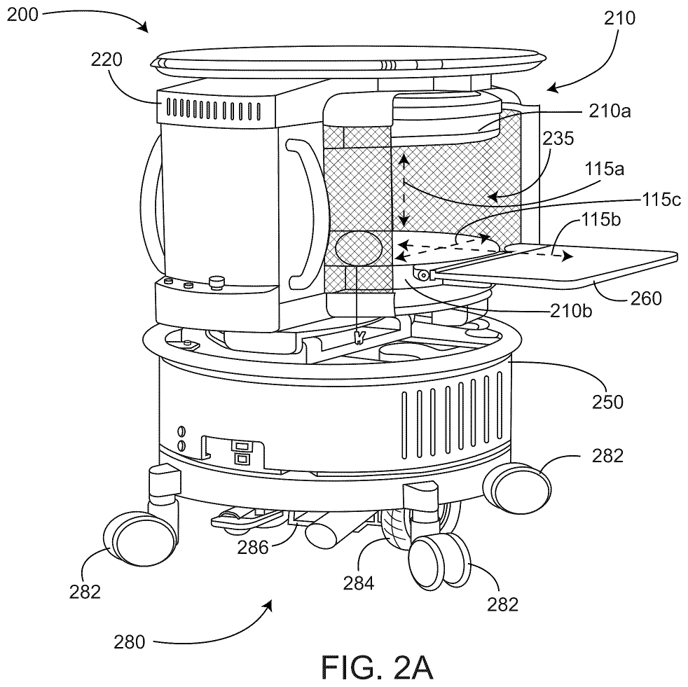

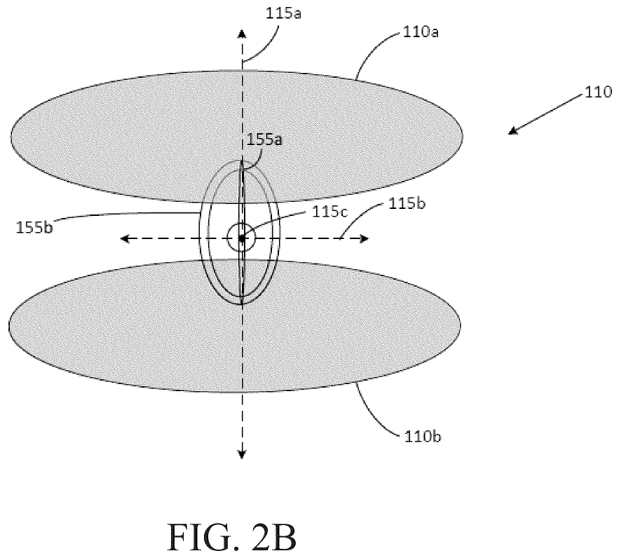

Following below are more detailed descriptions of various concepts related to, and embodiments of, an RF coil apparatus configured to operate in a low-field MRI system such as described above in connection with FIGS. 1-2A, though the technology described herein is not limited for use with any particular MRI system. It should be appreciated that the embodiments described herein may be implemented in any of numerous ways. Examples of specific implementations are provided below for illustrative purposes only. It should be appreciated that the embodiments and the features/capabilities provided may be used individually, all together, or in any combination of two or more, as aspects of the technology described herein are not limited in this respect.

FIG. 1 illustrates example components of an MRI system which may be used in conjunction with the RF coil apparatus described herein. In the illustrative example of FIG. 1, MRI system 100 comprises computing device 104, controller 106, pulse sequences repository 108, power management system 109, and magnetics components 120. It should be appreciated that MRI system 100 is illustrative and that an MRI system may have one or more other components of any suitable type in addition to or instead of the components illustrated in FIG. 1. Examples of MRI systems that may be used in accordance with some embodiments of the technology described herein are described in U.S. Pat. No. 10,222,434, filed Jan. 24, 2018 and issued Mar. 5, 2019, and titled “Portable Magnetic Resonance Imaging Methods and Apparatus”, each of which is incorporated by reference herein in their entireties.

As illustrated in FIG. 1, magnetics components 120 comprise B₀ magnets 122, shims 124, radio frequency transmit and receive coils 126, and gradient coils 128. B₀ magnets 122 may be used to generate the main magnetic field B₀. B₀ magnets 122 may be any suitable type or combination of magnetics components that can generate a desired main magnetic B₀ field. In some embodiments, B₀ magnets 122 may be one or more permanent magnets, one or more electromagnets, one or more superconducting magnets, or a hybrid magnet comprising one or more permanent magnets and one or more electromagnets and/or one or more superconducting magnets. In some embodiments, B₀ magnets 122 may be configured to generate a B₀ magnetic field having a field strength that is less than or equal to 0.2 T, within a range from 0.1 T to 0.2 T, within a range from 50 mT to 0.1 T, etc.

For example, in some embodiments, B₀ magnets 122 may include a first and second B₀ magnet, each of the first and second B₀ magnet including permanent magnet blocks arranged in concentric rings about a common center. The first and second B₀ magnet may be arranged in a bi-planar configuration such that the imaging region is located between the first and second B₀ magnets. In some embodiments, the first and second B₀ magnets may each be coupled to and supported by a ferromagnetic yoke configured to capture and direct magnetic flux from the first and second B₀ magnets.

Gradient coils 128 may be arranged to provide gradient fields and, for example, may be arranged to generate gradients in the B₀ field in three substantially orthogonal directions (X, Y, Z). Gradient coils 128 may be configured to encode emitted MR signals by systematically varying the B₀ field (the B₀ field generated by B₀ magnets 122 and/or shims 124) to encode the spatial location of received MR signals as a function of frequency or phase. For example, gradient coils 128 may be configured to vary frequency or phase as a linear function of spatial location along a particular direction, although more complex spatial encoding profiles may also be provided by using nonlinear gradient coils. In some embodiments, gradient coils 128 may be implemented using laminate panels (e.g., printed circuit boards), for example, as described in U.S. Pat. No. 9,817,093 filed Sep. 4, 2015 under Attorney Docket No.: O0354.70000US01 and titled “Low Field Magnetic Resonance Imaging Methods and Apparatus,” which is incorporated by reference herein in its entirety.

MRI is performed by exciting and detecting emitted MR signals using RF transmit and receive coils, respectively (often referred to as radio frequency coils), as described herein. The RF transmit/receive coils may include separate coils for transmitting and receiving, multiple coils for transmitting and/or receiving, or the same coils for transmitting and receiving. Thus, a transmit/receive component may include one or more coils for transmitting, one or more coils for receiving and/or one or more coils for transmitting and receiving. Transmit/receive coils are also often referred to as Tx/Rx or Tx/Rx coils to generically refer to the various configurations for the transmit and receive magnetics component of an MRI system. These terms are used interchangeably herein. In FIG. 1, RF transmit and receive coils 126 comprises one or more transmit coils that may be used to generate RF pulses to induce an oscillating magnetic field B₁. The transmit coil(s) may be configured to generate any suitable types of RF pulses. As described herein, the RF coils may comprise one or more primary RF coils and at least one secondary RF coil.

In some embodiments, transmit/receive coils comprise one or more coils configured to perform both transmit and receive operations during MR imaging. In some embodiments, transmit/receive coils comprise one or more separate coils, with one or more coils configured to perform transmit operations and one or more coils configured to perform receive operations during MR imaging. When separated, transmit coils may be fixed in a rigid configuration, such as coupled to a rigid frame, as described herein, to maximize the homogeneity of the magnetic field generated by the transmit coil. The receive coils may be flexible, such as fixed to a flexible substrate able to be wrapped around and/or positioned in close proximity to the patient anatomy being imaged in order to maximize SNR of detected MR signals. For example, the receive coils may be configured (e.g., shaped) for imaging a particular patient anatomy, such as a patient's knee, head, etc.

As described herein, one or more of the transmit and receive coils may be configured to be electronically and/or mechanically coupled to the MRI system. For example, one or more of the transmit and receive coils may be removably coupled to the MRI system such that the one or more of the transmit and receive coils may be coupled to and detached from the MRI system as desired.

Power management system 109 includes electronics to provide operating power to one or more components of the MRI system 100. For example, power management system 109 may include one or more power supplies, energy storage devices, gradient power components, transmit coil components, and/or any other suitable power electronics needed to provide suitable operating power to energize and operate components of MRI system 100. As illustrated in FIG. 1, power management system 109 comprises power supply system 112, power component(s) 114, transmit/receive circuitry 116, and thermal management components 118 (e.g., cryogenic cooling equipment for superconducting magnets, water cooling equipment for electromagnets).

Power supply system 112 includes electronics to provide operating power to magnetic components 120 of the MRI system 100. The electronics of power supply system 112 may provide, for example, operating power to one or more gradient coils (e.g., gradient coils 128) to generate one or more gradient magnetic fields to provide spatial encoding of the MR signals. Additionally, the electronics of power supply system 112 may provide operating power to one or more RF coils (e.g., RF transmit and receive coils 126, including RF transmit coil of the RF coil apparatus described herein) to generate and/or receive one or more RF signals from the subject. For example, power supply system 112 may include a power supply configured to provide power from mains electricity to the MRI system and/or an energy storage device. The power supply may, in some embodiments, be an AC-to-DC power supply configured to convert AC power from mains electricity into DC power for use by the MRI system. The energy storage device may, in some embodiments, be any one of a battery, a capacitor, an ultracapacitor, a flywheel, or any other suitable energy storage apparatus that may bidirectionally receive (e.g., store) power from mains electricity and supply power to the MRI system. Additionally, power supply system 112 may include additional power electronics encompassing components including, but not limited to, power converters, switches, buses, drivers, and any other suitable electronics for supplying the MRI system with power.

Amplifiers(s) 114 may include one or more RF receive (Rx) pre-amplifiers that amplify MR signals detected by one or more RF receive coils (e.g., coils 126), one or more RF transmit (Tx) power components configured to provide power to one or more RF transmit coils (e.g., coils 126 including RF receive coil of the RF coil apparatus described herein), one or more gradient power components configured to provide power to one or more gradient coils (e.g., gradient coils 128), and one or more shim power components configured to provide power to one or more shims (e.g., shims 124). In some embodiments the shim may be implemented using permanent magnets, electromagnetics (e.g., a coil), and/or a combination thereof. Transmit/receive circuitry 116 may be used to select whether RF transmit coils or RF receive coils are being operated.

As illustrated in FIG. 1, MRI system 100 includes controller 106 (also referred to as a console) having control electronics to send instructions to and receive information from power management system 109. Controller 106 may be configured to implement one or more pulse sequences, which are used to determine the instructions sent to power management system 109 to operate the magnetic components 120 in a desired sequence (e.g., parameters for operating the RF transmit and receive coils 126, parameters for operating gradient coils 128, etc.).

Examples of pulse sequences include zero echo time (ZTE) pulse sequences, balance steady-state free precession (bSSFP) pulse sequences, gradient echo pulse sequences, spin echo pulse sequences, inversion recovery pulse sequences, arterial spin labeling pulse sequences, diffusion weighted imaging (DWI) pulse sequences, Overhauser imaging pulse sequences, etc., aspects of which are described in U.S. Pat. No. 10,591,561 filed Nov. 11, 2015 under Attorney Docket No.: O0354.70002US01 and titled “Pulse Sequences for Low Field Magnetic Resonance,” which is incorporated by reference herein in its entirety.

As illustrated in FIG. 1, controller 106 also interacts with computing device 104 programmed to process received MR data. For example, computing device 104 may process received MR data to generate one or more MR images using any suitable image reconstruction process(es). Controller 106 may provide information about one or more pulse sequences to computing device 104 for the processing of data by the computing device. For example, controller 106 may provide information about one or more pulse sequences to computing device 104 and the computing device may perform an image reconstruction process based, at least in part, on the provided information.

Computing device 104 may be any electronic device configured to process acquired MR data and generate one or more images of a subject being imaged. In some embodiments, computing device 104 may be located in a same room as the MRI system 100 and/or coupled to the MRI system 100. In some embodiments, computing device 104 may be a fixed electronic device such as a desktop computer, a server, a rack-mounted computer, or any other suitable fixed electronic device that may be configured to process MR data and generate one or more images of the subject being imaged. Alternatively, computing device 104 may be a portable device such as a smart phone, a personal digital assistant, a laptop computer, a tablet computer, or any other portable device that may be configured to process MR data and generate one or images of the subject being imaged. In some embodiments, computing device 104 may comprise multiple computing devices of any suitable type, as aspects of the disclosure provided herein are not limited in this respect.

FIG. 2A illustrates a low power, portable low-field MRI system for which some embodiments of a RF coil apparatus is configured to operate in conjunction with. According to some embodiments, portable MRI system 200 is low-field MRI system operating with a B₀ magnetic field of less than or equal to 0.2 T and greater than 0.1 T, and according to some embodiments, portable MRI system 200 is a very low-field MRI system operating with a B₀ magnetic field of less than or equal to 0.1 T and greater than 10 mT (e.g., 0.1 T, 50 mT, 20 mT, etc.), that facilitate portable, low-cost, low-power MRI and may significantly increase the availability of MRI in a clinical setting. Portable MRI system 200 comprises a B₀ magnet 210 including at least one first permanent magnet 210a and at least one second permanent magnet 210b magnetically coupled to one another by a ferromagnetic yoke 220 configured to capture and channel magnetic flux to increase the magnetic flux density within the imaging region (field of view) of the MRI system. Permanent magnets 210a and 210b may be constructed using any suitable technique, (e.g., using any of the techniques, designs and/or materials described in the '434 patent previously incorporated by reference herein). Yoke 220 may also be constructed using any of suitable technique such as those described in the '434 patent. It should be appreciated that, in some embodiments, B₀ magnet 210 may be formed using electromagnets instead in addition to or as an alternative to permanent magnets (e.g., as also described in the '434 patent).

Exemplary B₀ magnet 210 illustrated in FIG. 2A is configured in a bi-planar arrangement such that the B₀ magnetic field is oriented along a vertical axis 115a. The direction of the B₀ magnetic field along the vertical axis for the exemplary configuration illustrated in FIG. 2A may be in either the upward or downward direction. As a result, radio frequency (RF) coils may have a principal axis aligned with a horizontal axis orthogonal to the vertical axis, such as longitudinal axis 115b or axial axis 115c. However, the B₀ magnetic field may be oriented along any suitable axis (e.g., along a horizontal axis, along a vertical axis, having components oriented along first and second axes substantially perpendicular to each other) and the RF coils may be oriented accordingly.

B₀ magnet 210 may be coupled to or otherwise attached or mounted to base 250 that, in addition to providing the load bearing structures for supporting the B₀ magnet, also includes an interior space configured to house electronics needed to operate portable MRI system 200. The exemplary portable MRI system 200 illustrated in FIG. 2A also comprises a conveyance mechanism 280 that allows the portable MRI system to be transported to different locations. The conveyance mechanism may comprise one or more components configured to facilitate movement of the portable MRI system, for example, to a location at which MRI is needed. According to some embodiments, conveyance mechanism comprises a motor 286 coupled to drive wheels 284. In this manner, conveyance mechanism 280 provides motorized assistance in transporting MRI system 200 to desired locations. Conveyance mechanism 280 may also include a plurality of castors 282 to assist with support and stability as well as facilitating transport.

MRI system 200 is also equipped with a fold-out bridge 260 that is capable of being raised (e.g., during transport) and lowered (e.g., as shown in FIG. 2A) to support patient anatomy during imaging, and may include any one or more of the features of a fold-out bridge described in U.S. Pat. Pub. No. 2020/0022612, titled “Patient Support Bridge Methods and Apparatus” and filed Jul. 19, 2019, the entirety of which is incorporated by reference herein. This exemplary low-field MRI systems can be used to provide point-of-care MRI, either by bringing the MRI system directly to the patient or bringing the patient to a relatively nearby MRI system (e.g., by wheeling the patient to the MRI system in a standard hospital bed, wheelchair, etc.). The inventors have a radio frequency coil apparatus configured for use in such an MRI system, though the aspects are not limited for use with any particular MRI system.

FIG. 2B illustrates an exemplary bi-planar B₀ magnetic configuration and radio frequency coils of the example MRI system of FIG. 2A configured to generate radio frequency signals, in accordance with some embodiments of the technology described herein. As described herein, MRI involves placing a subject to be imaged (e.g., all or a portion of patient anatomy) in a static, homogenous magnetic field B₀ to align a subject's atomic net magnetization (often represented by a net magnetization vector) in the direction of the B₀ field. One or more transmit coils are then used to generate a pulsed magnetic field B₁ having a frequency related to the rate of precession of atomic spins of the atoms in the magnetic field B₀ to cause the net magnetization of the atoms to develop a component in a direction transverse to the direction of the B₀ field. After the B₁ field is turned off, the transverse component of the net magnetization vector precesses and its magnitude decays over time until the net magnetization re-aligns with the direction of the B₀ field if allowed to do so. This process produces MR signals that can be detected, for example, by measuring electrical signals induced in one or more receive coils of the MRI system that are tuned to resonate at the frequency of the MR signals.

MR signals are rotating magnetic fields, often referred to as circularly polarized magnetic fields, that can be viewed as comprising linearly polarized components along orthogonal axes. That is, an MR signal is composed of a first sinusoidal component that oscillates along a first axis and a second sinusoidal component that oscillates along a second axis orthogonal to the first axis. The first sinusoidal component and the second sinusoidal component oscillate 900 out-of-phase with each other. An appropriately arranged coil tuned to the resonant frequency of the MR signals can detect a linearly polarized component along one of the orthogonal axes. In particular, an electrical response may be induced in a tuned receive coil by the linearly polarized component of an MR signal that is oriented along an axis approximately orthogonal to the current loop of the coil, referred to herein as the principal axis of the coil.

Accordingly, radio frequency coils configured to excite and detect MR signals, which may include separate coils for transmitting and receiving, multiple coils for transmitting and/or receiving, or the same coils for transmitting and receiving, need to be oriented appropriately relative to the B₀ magnetic field to perform MRI. Whereas conventional high-field MRI scanners produce a B₀ field oriented in directions along a horizontal axis (e.g., along the longitudinal axis of the bore), exemplary low-field MRI systems described herein may produce a B₀ field oriented in directions along a vertical axis. For example, FIG. 2B illustrates an exemplary bi-planar geometry for a B₀ magnet, in accordance with some embodiments. B₀ magnet 110 is schematically illustrated by magnet 110a and 100b arranged substantially parallel to one another to generate a B₀ field generally along axis 115a (either in the upward or downward direction) to provide a field of view between the magnets 110a and 100b (i.e., a region between the magnets wherein the homogeneity of the B₀ field is suitable for MRI).

A first RF coil (or multiple RF coils) is schematically illustrated as RF coil 155a, which is/are arranged to generate a pulsed oscillating magnetic field generally along axis 115b (i.e., the principal axis of RF coil(s) 155a) to stimulate an MR response and/or to detect the MR signal component oriented substantially along the principal axis 115b (i.e., linearly polarized components of the MR signal aligned with the coil's principal axis). A second RF coil (or multiple RF coils) is schematically illustrated as RF coil 155b, which is/are arranged to generate a pulsed oscillating magnetic field generally along axis 115c (i.e., the principal axis of RF coil(s) 150c into and out of the plane of the drawing) to stimulate an MR response and/or to detect the MR signal component oriented substantially along the principal axis 115c (i.e., linearly polarized components of the MR signal aligned with the coil's principal axis).

### Example Radio Frequency Coil Apparatuses

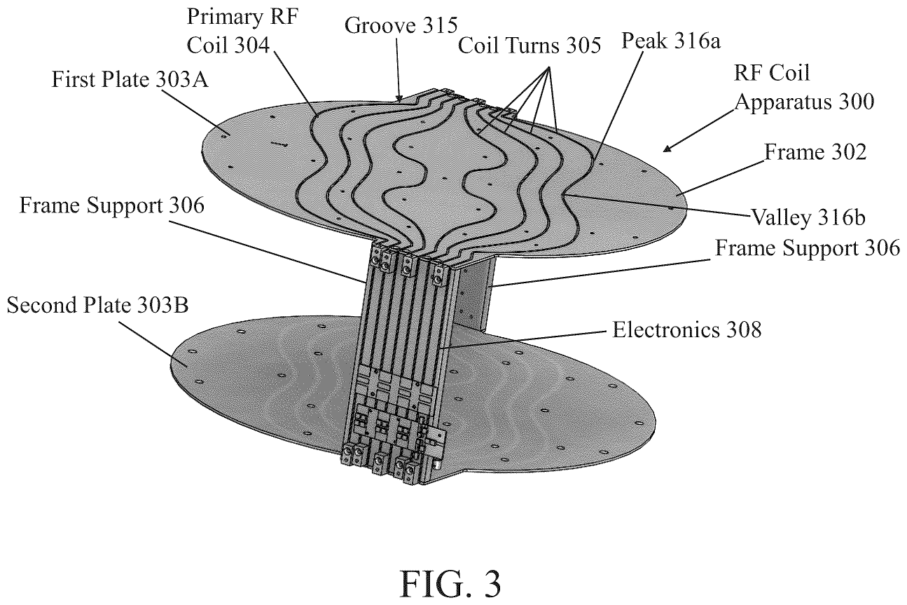

FIG. 3 illustrates an example RF coil apparatus, in accordance with some embodiments of the technology described herein. As shown in FIG. 3, RF coil apparatus 300 comprises a primary RF coil 304. Although one primary RF coil 304 is shown in FIG. 3, in some embodiments, the RF coil apparatus 300 may comprise multiple primary RF coils. As described herein, the primary RF coil 304 generates a first B₁ magnetic field.

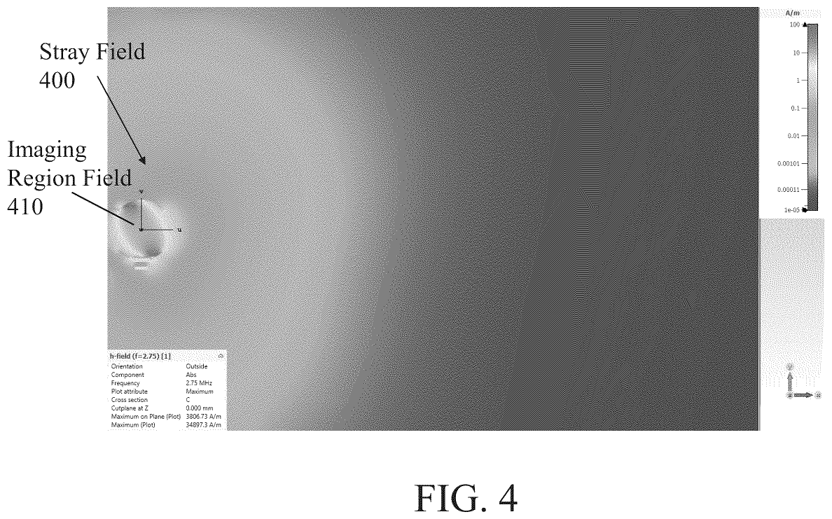

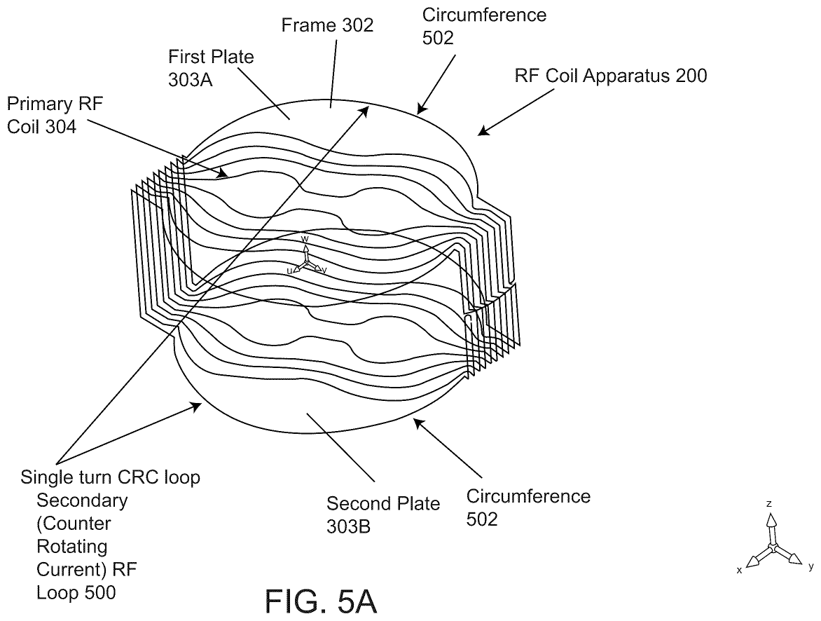

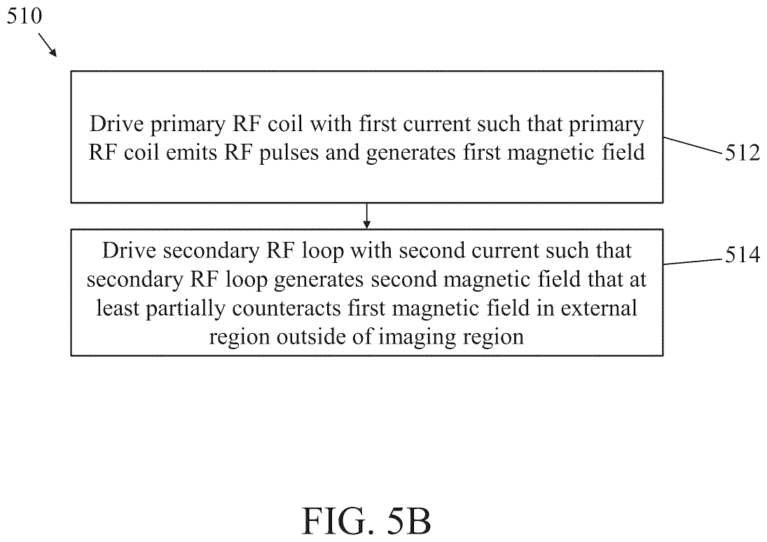

As described herein, RF coil apparatus further comprises at least one secondary RF coil for reducing and/or canceling a stray field generated by the primary RF coil. For the sake of illustration, the RF coil apparatus shown in FIG. 3 does not illustrate secondary RF coil. For example, FIG. 4, described further herein, illustrates the stray magnetic field generated by primary RF coil 304 when no secondary RF coil is implemented. Aspects of the secondary RF coil are further illustrated beginning at FIG. 5A, for example.

RF coil apparatus 300 may comprise a frame 302. Frame 302 comprises a first plate 303A and a second plate 303B. Second plate 303B is disposed opposite the first plate 303A. An imaging region for receiving and imaging a patient anatomy may be formed in the space between the first and second plates 303A-B. In the illustrated embodiment of FIG. 3, the first and second plates 303A-B are substantially parallel to each other which may enhance the homogeneity of the magnetic field generated by coil 304, as described herein.

The primary RF coil 304 is wound around frame 302 in the illustrated embodiment. The B1 field generated by the primary RF coil 304 extends in an imaging region between first and second plates 303A-B of the frame 302. However, the magnetic field generated by the primary RF coil 304 further extends beyond the imaging region, to an external region outside the imaging region at least in part due to openings in the frame (e.g., between the first and second plates 303A-B.

Frame 302 may be made of any suitable material. For example, frame 302 may be made of a non-ferrous material, such as plastic (e.g., Kydex). Frame 302 may be rigid, to prevent deformation of the frame 302. Use of a rigid frame 302 assists in enhancing the stability of the homogeneous magnetic field generated by primary RF coil 304. In particular, an important design criteria for RF transmit coils is configuring the coil to be wound about a rigid frame to ensure the homogeneity of the magnetic field generated by the coil is optimized and remains constant as opposed to fluctuating as may occur in a flexible coil.

In the illustrated embodiment, first and second plates 303A-B are disk-shaped, however other suitable shapes are possible, and aspects of the technology are not limited in this respect. The first and second plates 303A-B may be substantially the same size, in some embodiments (e.g., having a diameter of approximately 60 cm).

Frame 302 further comprises supports 306. Supports 306 separate first and second plates 303A-B to form an imaging region therebetween, as described herein. The supports 306 may separate first and second plates 303A-B such that imaging region comprises a spherical volume having a radius of approximately 10 cm. In some embodiments, the spherical volume comprises a radius of at least 5 cm, 6 cm, 7 cm, 8 cm, 9 cm, 10 cm, etc. For example, supports have a height of approximately 30 cm each. In the illustrated embodiment, the frame comprises two supports, however other configurations are possible (e.g., 1 support, 3 supports, etc.). In the illustrated embodiment of FIG. 3, supports 306 are c-shaped.

Primary RF coil 304 and secondary RF loop 500 described herein may comprise at least one conductor. In some embodiments, primary RF coil 304 and/or secondary RF loop 500 comprises a plurality of conductors. In such embodiments, the plurality of conductors may be connected together in series at capacitive junctions.

The pattern of windings of the primary RF coil 304 shown in FIG. 3 is a result of optimizing the uniformity of the magnetic field generated by the primary RF coil 304. The optimization is based on an assumption that RF current magnitude and phase are the same for all turns of the coil. To ensure that this assumption is true, the conductors of the coil (and as a result, the coil turns) are disposed in series. Configuring the conductors and turns in a parallel configuration introduces problems in ensuring current is split evenly between the turns of the coil.

In some embodiments, conductor(s) of the primary RF coil 304 and/or the secondary RF loop 500 comprise Litz wire. Multi-stranded Litz wire comprises thousands of wires bound together in a braid. Current is split between the multiple wires thereby decreasing current density in each strand of wire. The distribution of current between the wires increases gain, in turn increasing efficiency of the circuit formed by the wire (by increasing the Quality factor (Q) of the circuit).

In some embodiments, conductor(s) of primary RF coil 304 and/or the secondary RF loop 500 comprise copper wire. The resistance of a wire is inversely proportional to the radius of the wire. Therefore, copper wires having increased diameter may be used in some embodiments to achieve a target series resistance.

In some embodiments, RF coil apparatus 300 may be configured to operate in the low-field regime, for example, in combination with a low-field MRI system as described herein. In such embodiments where the RF coil apparatus 300 is configured to operate in the low-field regime, primary RF coil 304 may have significantly larger dimensions of uninterrupted conductor length than conventional RF coils configured to operate in the high-field regime due to the lower resonant frequency involved in low-field MRI, as discussed above. For example, the primary RF coil 304 may have a length of approximately 14 meters. In some embodiments, the coil comprises a length of at least 5 meters, at least 10 meters, at least 11 meters, at least 12 meters, at least 13 meters, at least 14 meters, etc. It should be appreciated that the specific dimensions illustrated are exemplary and may be chosen to be smaller or larger.

As described herein, the primary RF coil 304 may be constructed from one or more conductors. The conductor(s) may be wound around the frame 302 in a plurality of turns 305. For example, the frame 302 may comprise a plurality of grooves 315 in which conductors of the primary RF coil 304 are disposed. For example, as shown in FIGS. 3B-3C, each of the first plate 303A and the second plate 303B comprise multiple grooves 315. Each of the plurality of grooves 315 may comprise a path which extends around the frame 302 along each of the first and second plates 303A-B. Each path may form a substantially closed loop. One turn of the primary RF coil may comprise the length of the primary RF coil which is disposed in a single path formed by a groove of the plurality of grooves 315. Therefore, one turn extends along each of the first and second plates 303A-B and forming a substantially closed loop interrupted only by electronics 308 of the RF coil apparatus 300, as described herein. In some embodiments, each conductor of the plurality of conductors may be disposed in a one of the plurality grooves and comprising one of the plurality of turns. In some embodiments, each turn comprises multiple conductors.

Each conductor may have a length of approximately 180 cm. The conductor length may comprise a conductive path uninterrupted by electronics such as capacitors, as described herein. The conductors may be coupled together at capacitive junctions comprising at least one capacitor, as described herein. The length of a conductor between respective capacitive junctions may comprise a turn. As such, each turn of the primary RF coil 304 may have a length of approximately 180 cm. In some embodiments, each conductor and/or each turn has a length of at least 100 cm, at least 150 cm, at least 175 cm, etc.

The number of turns may be designed to maximize the efficiency of the primary RF coil 304. The efficiency of the coil 304 may be referred to as the power needed to generate a particular magnetic field. The Quality factor (Q) which is indicative of efficiency may be represented by the ratio of reactance to resistance. The reactance of the primary RF coil increases with the square of the number of turns of the primary RF coil while the resistance of the primary RF coil increases linearly with the number of turns. Therefore, increasing the number of turns increases Q which increases efficiency.

The number of turns of the primary RF coil 304 may be optimized, as the inventors have recognized that increasing the number of turns of the primary RF coil can have diminishing returns. Specifically, the increase in efficiency decreases with each additional turn of the primary RF coil. In some embodiments, the plurality of turns comprises at least six turns. In some embodiments, the plurality of turns comprises no more than 12 turns. In the illustrated embodiment of FIGS. 3A-3C, the plurality of turns 305 comprises 8 turns. However, any number of turns can be selected and the optimal number of turns may depend on the geometry of the RF coil apparatus (e.g., the type of patient anatomy the apparatus is configured to surround), the type of conductor and method of fabrication and/or desired operating characteristics of the primary RF coil.

The plurality of grooves 315 may be shaped non-linearly, as shown in the illustrated embodiments of FIG. 3. For example, at least one groove of the plurality of grooves 315 may be undulating, having multiple (e.g., at least two) undulation points 316a, 316b (e.g., the undulation points comprising a peak or a valley). In some embodiments, at least one groove of the plurality of grooves comprises one or more peaks 316a and one or more valleys 316b. As shown in FIG. 3, a peak 316a may refer to a point on a groove 315 that is a local maximum and a valley may refer to a point on a groove that is a local minimum. In some embodiments, as shown in FIG. 3, the undulation of at least some of the plurality of grooves 315 (e.g., the position, amount, and/or height of the one or more peaks and valleys) may differ from others of the plurality of grooves 315.

The undulation of the grooves 315 may be chosen to optimize the homogeneity of the magnetic field. For example, the pattern of grooves 315 may be chosen with the overall goal of optimizing the uniformity of the magnetic field generated by the RF transmit coil. Deviation from a target magnetic field uniformity may be measured over the volume of the imaging region for each fragment of the RF coil. The orientation of each coil fragment may be chosen such that deviation from the target magnetic field is minimized, resulting in a pattern of peaks 315a and valleys 316b.

A flexible or rigid RF receive coil may be used with the transmit coils of the RF coil apparatus 300 described herein, including the example RF receive coils described in U.S. patent application Ser. No. 15/152,951 filed May 12, 2016 titled “Radio Frequency Coil Methods and Apparatus” and U.S. patent application Ser. No. 15/720,245 filed Sep. 29, 2017 titled “Radio Frequency Coil Tuning Methods and Apparatus”, each of which are incorporated by reference herein in their entireties.

FIG. 4 illustrates an example graph of a stray field generated by the example RF coil apparatus of FIG. 3, in accordance with some embodiments of the technology described herein. As described herein, the primary RF coil of the RF coil apparatus generates a first magnetic field (e.g., the B1 magnetic field) that extends not only in the imaging region of an MRI system but also to an external region outside of the imaging region. Therefore, as shown in FIG. 4, the first magnetic field generated by the primary RF coil comprises a first portion labeled the imaging region field 410 which extends across the imaging region of the MRI system as well as the stray field 400 in the external region outside the imaging region.

FIG. 4 illustrates that the strength of the stray field 400 component of the first magnetic field is relatively high. As described herein, high strength stray fields may have deleterious effects of nearby electronic devices, including nearby MRI systems, due to the electromagnetic interference caused by the stray field. In addition, some regulations require the strength of the stray field to remain under a certain limit.

Accordingly, the inventors have developed an RF coil apparatus which further comprises a secondary RF coil that at least partially counteracts the stray field generated by the primary RF coil. The secondary RF coil may also be described herein as a counter rotating current (CRC) coil. FIG. 5A illustrates an example of an RF coil apparatus comprising a counter rotating current coil, in accordance with some embodiments of the technology described herein.

In particular, FIG. 5A illustrates the RF coil apparatus 300 shown in FIG. 3 having a secondary RF loop 500. As described above, in some embodiments, the secondary RF coil described herein may be implemented as an additional loop to the primary RF coil. One such embodiment is shown in FIG. 5A. That is, the secondary RF coil and the primary RF coil may be implemented as a single coil having a secondary loop to the primary coil that generates a secondary magnetic field to counteract the primary magnetic field generated by the primary RF coil in the external region outside of the imaging region, as described herein. In other embodiments, as described herein, the secondary RF coil may comprise a coil that is separate from the primary RF coil.

In the illustrated embodiment of FIG. 5A, the secondary RF loop 500 is wound around a circumference of the first and second plates 303A-B. The secondary RF loop 500 generates a second magnetic field in an external region outside of the imaging region of the MRI system. The second magnetic field counteracts the first magnetic field generated by the primary RF coil 304. Accordingly, an effective strength of the magnetic field in the external region outside of the imaging region of the MRI system is reduced and/or canceled.

As described herein, in some embodiments, the secondary RF coil may be configured such that a current in the secondary RF coil is 180 degrees out of phase with a current in the primary RF coil. That is, if the current in the primary RF coil runs clockwise, the secondary RF loop may be configured having current that runs counterclockwise. As such, the secondary RF loop is referred to herein as a “counter rotating current” loop or coil. The counter rotating current in the secondary RF loop allows for generation of the second magnetic field that counteracts the first magnetic field generated by the primary RF coil.

Controlling the current in the secondary RF coil to be 180 degrees out of phase with the current in the primary RF coil is possible where the secondary RF coil and the primary RF coil are controlled by independent drive circuits. In other embodiments, such as the embodiment illustrated in FIG. 5A, however, the secondary RF coil and the primary RF coil may be electrically connected such that the phases of the current in both the primary and secondary RF loops are equal. In such embodiments, the pattern in which the secondary RF loop is wound relative to the primary RF coil allows for generation of the second magnetic field with the secondary RF loop that counteracts the first magnetic field generated by the primary RF coil in the external region outside of the imaging region.

The secondary RF loop 500 may be oriented to optimize the ability of the secondary RF loop 500 to counteract stray field. For example, the secondary RF loop 500 is oriented such that components of the second magnetic field generated by the secondary RF loop 500 are oriented along the same axes as components of the first magnetic field generated by the primary RF coil. That is, if the first magnetic field comprises a first component along the x-axis and a second component along the y-axis, the secondary RF loop may be oriented such that the second magnetic field has components along the x- and y-axes (e.g., a first axis and a second axis substantially perpendicular to the first axis). In other embodiments, the second magnetic field generated by the secondary RF loop may be directed substantially along a single axis.

In some embodiments, the secondary RF loop comprises multiple loops (e.g., multiple coils), as described further herein. For example, the secondary RF loop may comprise a first secondary RF loop and a second secondary RF loop. Any suitable number of secondary RF loops may be implemented.

In some embodiments, where the secondary RF loop comprises multiple loops, the respective secondary RF loops may be oriented such that each secondary RF loop generates a portion of the second magnetic field along a respective axis. For example, a first secondary RF loop may generate a first component of the second magnetic field along a first axis and a second secondary RF loop may generate a second component of the second magnetic field along a second axis. The second axis may be substantially perpendicular to the first axis.

In some embodiments, the secondary RF loop may be tilted with respect to the x- and y-axes. For example, where the first magnetic field comprises components along first and second perpendicular axis, the secondary RF loop may be tilted with respect to the first and second perpendicular axes such that a single secondary RF loop generates components along each of the first and second perpendicular axes.

The secondary RF loop 500 may be positioned such that the second magnetic field does not significantly counteract the first magnetic field present in the imaging region. For example, in the illustrated embodiments, the secondary RF loop may be disposed outside of or on an edge of the imaging region. In some embodiments, the strength of the first magnetic field in the imaging region may be much greater than the strength of the second magnetic field in the imaging region such that any counteraction of the second magnetic field on the first magnetic field in the imaging region is negligible.

The secondary RF loop 500 may be controlled and/or powered with electronics (e.g., a power source). The electronics may be the same or different than the electronics that control and/or power primary RF coil (e.g., electronics 308). For example, where the secondary RF loop is in series with the primary RF coil, the primary RF coil and the secondary RF loop share a single drive circuit. In embodiments where the secondary RF loop 500 comprises multiple loops (e.g., multiple coils), the respective loops of the secondary RF loop 500 may be controlled and/or powered with the same or different electronics.

As shown in the illustrated embodiment of FIG. 5A, the secondary RF loop 500 is in series with the primary RF coil 304. In some embodiments, the secondary RF loop 500 may be disposed within an RF shield (not shown) of the MRI system. The RF shield may also house the primary RF coil 304. The secondary RF loop 500 is wound along the circumference 502 of the first and second plates 303A-B. Distancing the secondary RF loop 500 from the isocenter of the first and second plates 303A-B reduces the current and power necessary to drive the secondary RF loop 500. The illustrated embodiment of FIG. 5A may achieve a reduction of stray field strength of at least 2.5 dB.

FIG. 5B illustrates an example method of operating an RF coil apparatus comprising a CRC coil, in accordance with some embodiments of the technology described herein. The method 510 may begin at act 512, wherein the primary RF coil is driven with a first current. Driving the primary RF coil with the first current causes the primary RF coil to emit RF pulses and to generate the first magnetic field. As described herein, the first magnetic field generated by the primary RF coil extends both in the imaging region of the MRI system and beyond, to an external region outside of the imaging region.

At act 514, a secondary RF loop may be driven with a second current. Driving the secondary RF loop with the second current may cause the secondary RF loop to generate a second magnetic field that at least partially counteracts the first magnetic field in the external region outside of the imaging region. That is, the effective magnetic field which comprises the superposition of the first and second magnetic fields in the external region outside of the imaging region is reduced.

In some embodiments, as described herein, the second current may be 180 degrees out of phase with the first current in order to achieve reduction and/or cancellation of the first magnetic field in the external region (e.g., the stray field). For example, where the primary RF coil and the secondary RF coil use separate drive circuits, the respective coils may be controlled with different currents. In other embodiments, as described herein, the secondary RF coil may be in series with the primary RF coil and controlled by a same drive circuit. In such cases, the first and second currents may be equal.

As described herein, the secondary RF loop may comprise a secondary RF coil that is separate from the primary RF coil. In other embodiments, the secondary RF loop and the primary RF coil may be a single coil comprising the secondary RF loop.

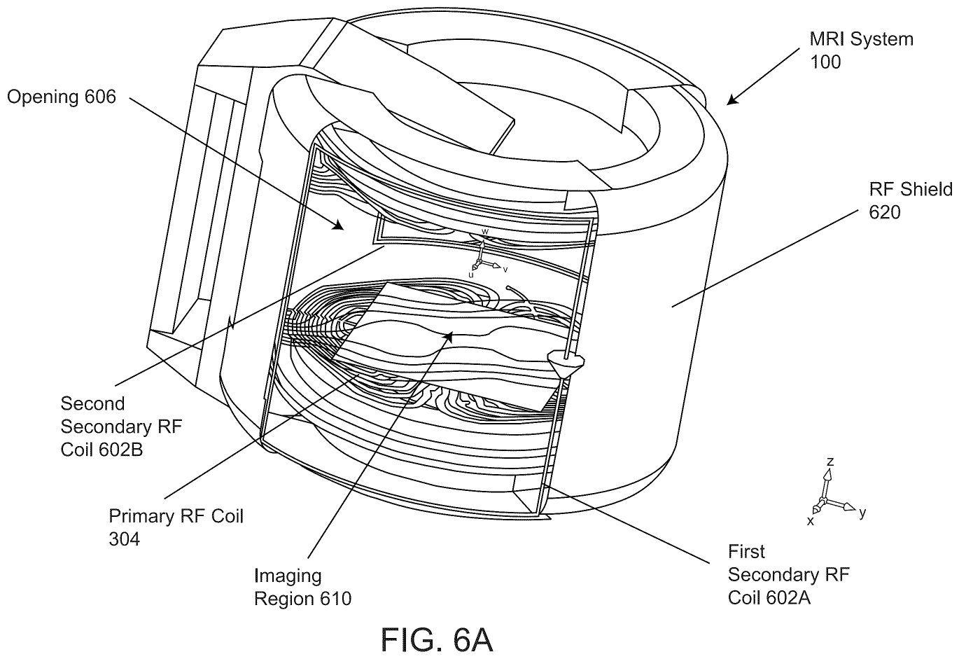

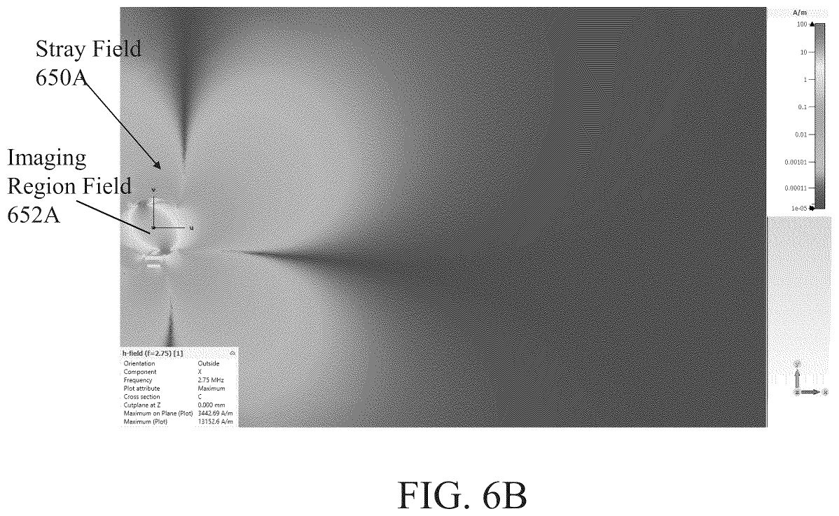

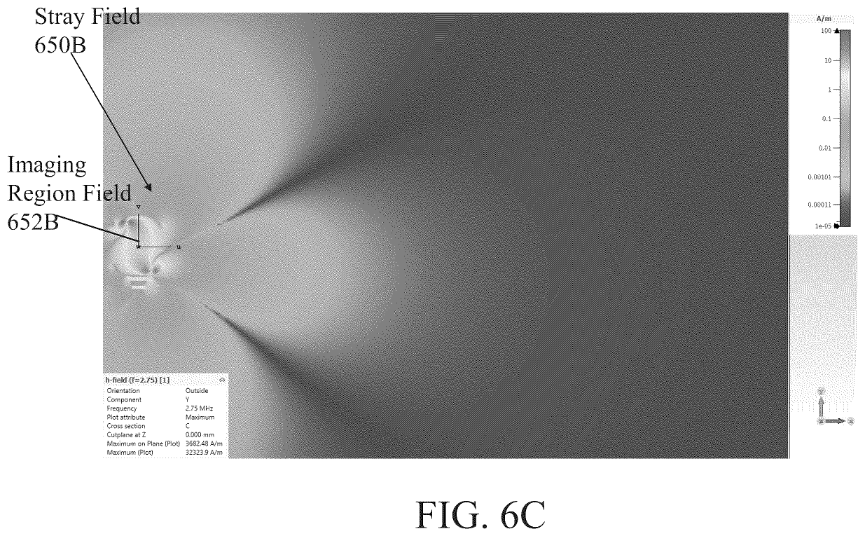

FIG. 6A illustrates another example RF coil apparatus comprising a CRC coil, in accordance with some embodiments of the technology described herein. Secondary RF coil 602A, 602B may be generally of the same type as secondary RF loop 500, for example, having all or some of the same features describes herein with respect to secondary RF loop 500. In the illustrated embodiment of FIG. 6A, the primary RF coil 304 and the secondary RF coil 602A, 602B are no longer implemented in series, however. In this embodiment, the secondary RF coil 602A, 602B comprise separate coils from the primary RF coil 304.

In the illustrated embodiment, the secondary RF coil 602A, 602B comprises multiple coils. In particular, the secondary RF coil 602A, B comprises a first secondary RF coil 602A and a second secondary RF coil 602B.

As shown in FIG. 6A, the MRI system 100 comprises a RF shield 620. The RF shield 620 may at least partially encase one or more components of the MRI system 100 (e.g., one or more B0 magnets, the primary RF coil). RF shield 620 may reduce magnetic field emissions from the imaging region to the external region outside of the imaging region. However, RF shield 620 may not completely attenuate the stray field in the external region outside of the imaging region.

The RF shield 620 may comprise at least one opening 606. The opening 606 may allow for insertion of patient anatomy into the imaging region 610. As described herein, the opening(s) 606 increase the amount of the first magnetic field that extends beyond the imaging region 610, thereby increasing the stray field. In the illustrated embodiment, the RF shield 620 comprises two openings 606 disposed on opposite sides of the imaging region 610. Each of the first and second secondary RF coils 602A, 602B sit in a respective opening 606. In some embodiments, a loop of a respective secondary RF coil may have an area that is greater than an area of the opening. Although in the illustrated embodiment two openings are shown, in some embodiments one or more than two openings may be implemented having a secondary RF coil disposed therein.

In some embodiments, the secondary RF coil is disposed inside the RF shield. In some embodiments, the secondary RF coil is disposed at least partially inside and at least partially outside the RF shield. In some embodiments, the secondary RF coil is disposed outside the RF shield. In embodiments where the secondary RF loop is disposed outside of the RF shield, the RF shield may prevent the secondary magnetic field generated by the secondary RF loop from reaching the imaging region thereby preventing the secondary magnetic field from reducing the magnetic field in the imaging region.

In the illustrated embodiment, the first and second secondary RF coils 602A, 602B are oriented such that the second magnetic field generated by the secondary RF coils is oriented along the x-axis. The first and secondary RF coils of the illustrated embodiment can achieve a reduction of the stray field of at least 1.5 dB.

FIGS. 6B-6C illustrate example graphs of the stray field generated by the example RF coil apparatus of FIG. 6A, in accordance with some embodiments of the technology described herein. In particular, FIG. 6B illustrates the relative strengths of the portions of the imaging region field 652A and the stray field 650A oriented along the x-axis. FIG. 6C illustrates the relative strengths of the portions of the imaging region field 652B and the stray field 650B oriented along the y-axis. The portion of the stray field oriented along the x-axis is reduced by operation of the secondary RF coil of FIG. 6A given the orientation of the secondary RF coils along the x-axis.

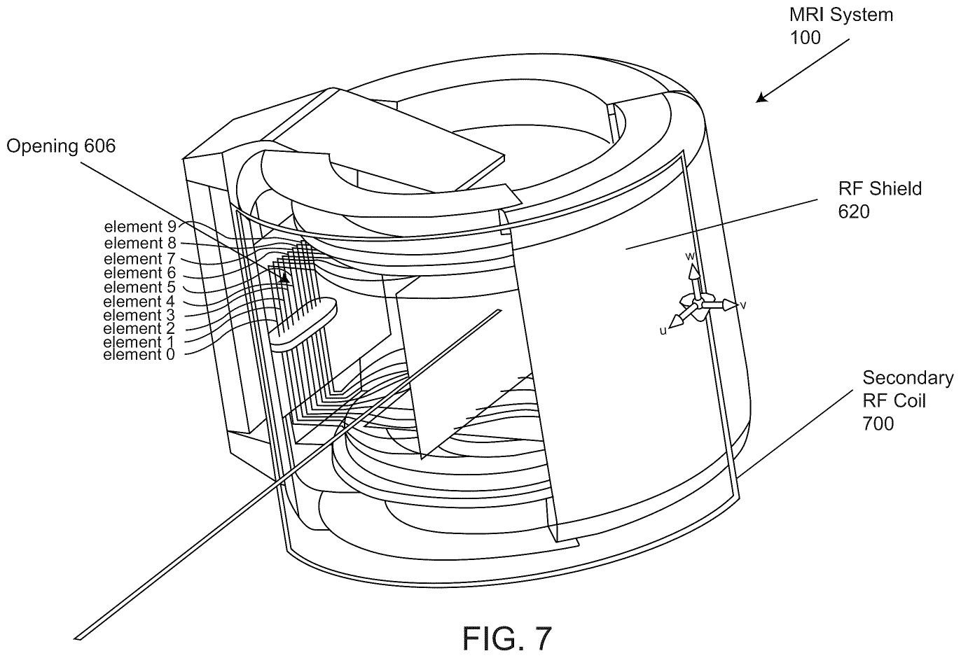

FIG. 7 illustrates another RF coil apparatus comprising a CRC coil, in accordance with some embodiments of the technology described herein. Secondary RF coil 700 may be generally of the same type as secondary RF loop 500, for example, having all or some of the same features describes herein with respect to secondary RF loop 500. However, in the illustrated embodiment of FIG. 7, the secondary RF coil 700 comprises a separate coil from the primary RF coil 304.

In the illustrated embodiment of FIG. 7, the secondary RF coil 700 is offset with respect to the opening 606 and is disposed outside of RF shield 620. Accordingly, the second magnetic field does not detract from the first magnetic field inside the imaging region.

The secondary RF coil 700 comprises a single coil which is rotated over the z-axis and therefore tilted relative to the x-axis. Accordingly, the secondary RF coil 700 generates a second magnetic field having components directed along two substantially perpendicular axes (e.g., x- and y-axes). The secondary RF coil 700 is capable of reducing the stray field by at least 2.2 dB.

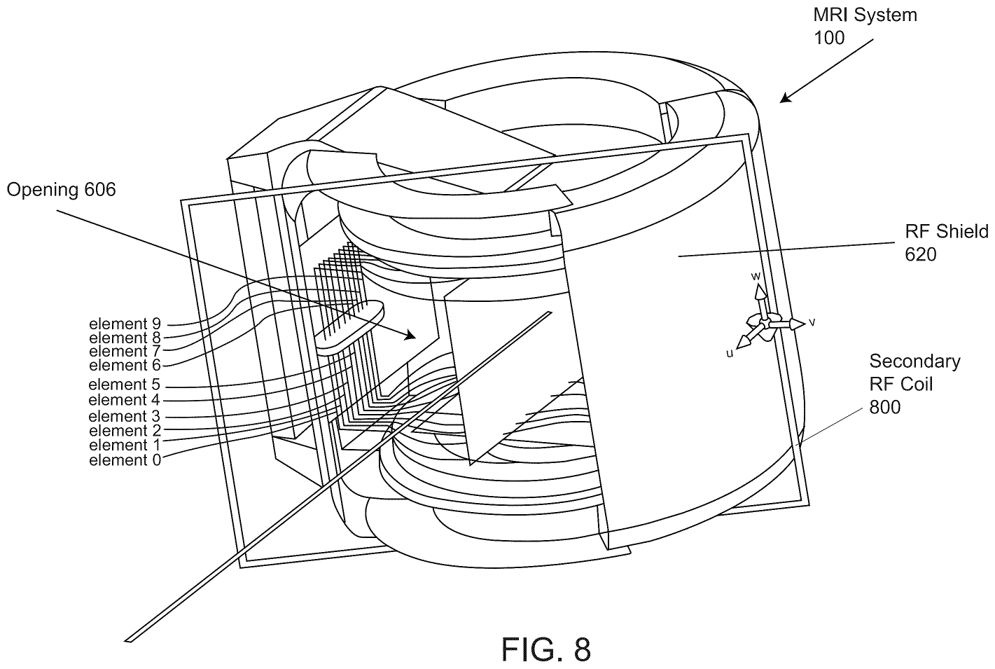

FIG. 8 illustrates another example RF coil apparatus comprising a CRC coil, in accordance with some embodiments of the technology described herein. Secondary RF coil 800 may be generally of the same type as secondary RF coil 700, for example, having all or some of the same features describes herein with respect to secondary RF coil 700.

The secondary RF coil 800 is tilted relative to the x-axis. Accordingly, the secondary RF coil 800 generates a second magnetic field having components directed along two substantially perpendicular axes (e.g., x- and y-axes). The secondary RF coil 800 is capable of reducing the stray field by at least 2.2 dB.

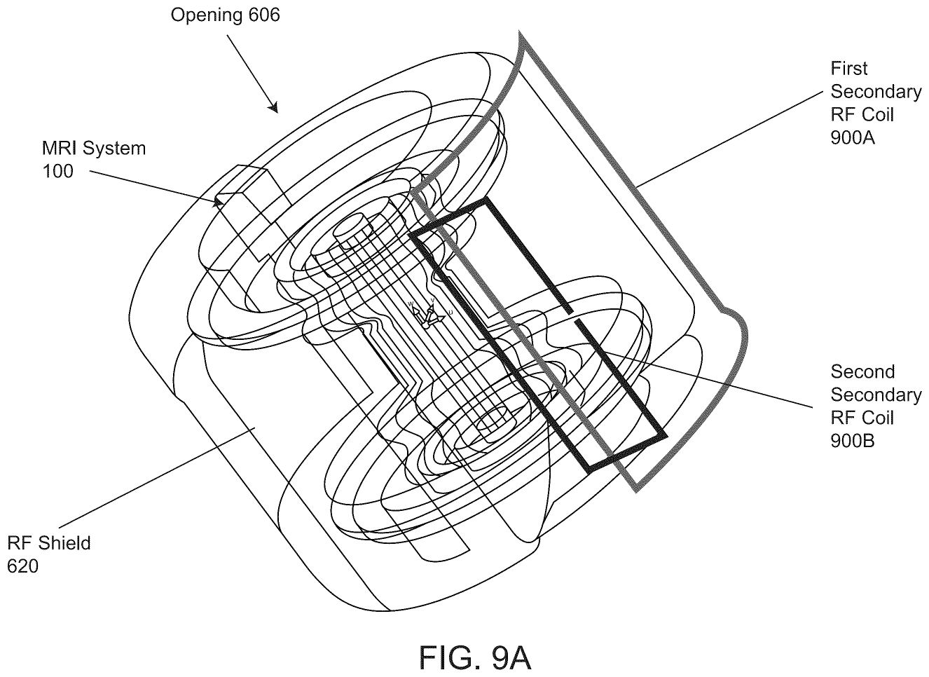

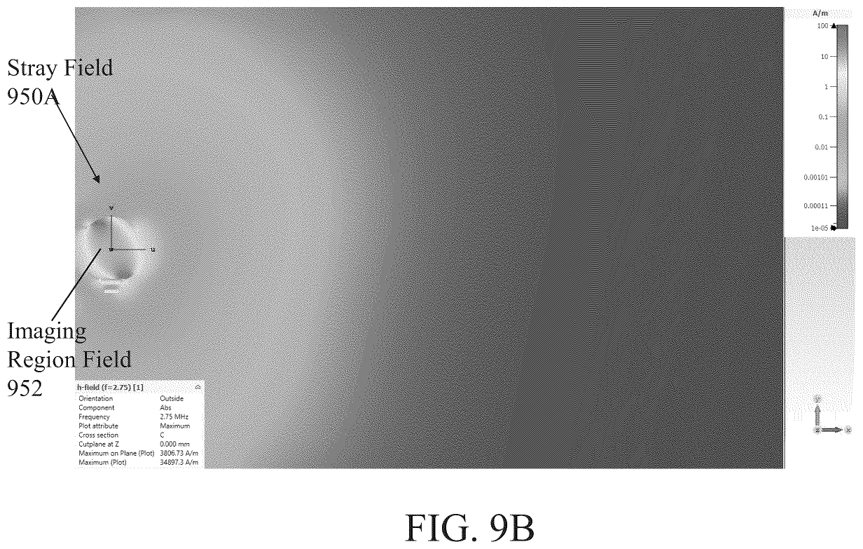

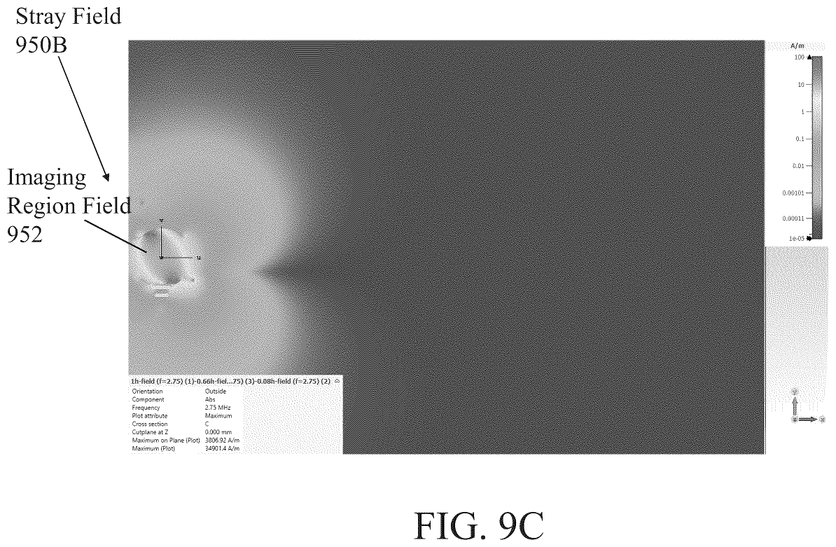

FIG. 9A illustrates another example RF coil apparatus comprising a CRC coil, in accordance with some embodiments of the technology described herein. The secondary RF coil 900A, 900B may be generally of the same type as secondary RF coils 602A, 602B for example, having all or some of the same features describes herein with respect to secondary RF coils 602A, 602B.

In the illustrated embodiments, the RF coil apparatus comprises a first secondary RF coil 900A and a second secondary RF coil 900B. Together, the first and second secondary RF coils 900A, 900B counteract both x- and y-axis directed components of the first magnetic field. In the illustrated embodiment, the first secondary RF coil 900A is oriented to generate a portion of the second magnetic field along the x-axis. The second secondary RF coil 900B is oriented to generate a portion of the second magnetic field along the y-axis. The second secondary RF coil 900B may be oriented along the xz plane.

In the illustrated embodiment, the first secondary RF coil 900A is offset relative to the opening 606 of the RF shield 620. In some embodiments, one or both of the first and second secondary RF coils 900A, 900B may be disposed inside or at least partially within the RF shield. In some embodiments, one or both of the first and second secondary RF coils 900A, 900B may be disposed outside of the RF shield 620.

As described herein, the current in the secondary RF coil may be 180 degrees out of phase with a current of the primary RF coil. Current in the respective first and second secondary RF coils may be independently controlled. For example, each of the primary and secondary RF coils may be controlled by respective drive circuits.

Although some embodiments have been illustrated showing multiple secondary RF coils, it should be appreciated that in some embodiments, only one of the illustrated secondary RF coils may be implemented.

FIGS. 9B-9C illustrates example graphs of the stray field generated by the example RF coil apparatus of FIG. 9A, in accordance with some embodiments of the technology described herein. FIG. 9B illustrates the relative strengths of the portions of the imaging region field 952A and the stray field 950B oriented along the x-axis. FIG. 9C illustrates the relative strengths of the portions of the imaging region field 952A and the stray field 950B oriented along the y-axis. The secondary RF coil 900A, 900B is capable of reducing the stray field by at least 55 dB.

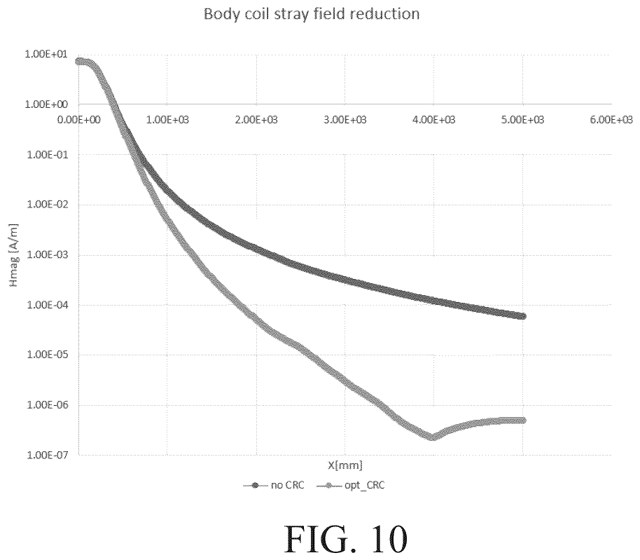

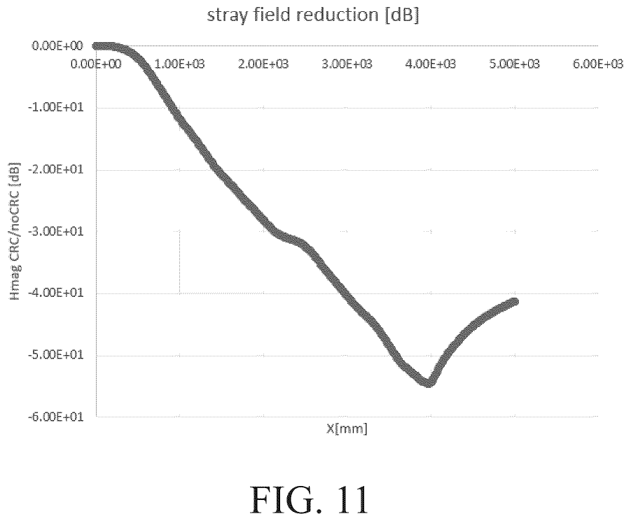

FIG. 10 illustrates a graph comparing the relative stray field reduction with and without use of a CRC coil, in accordance with some embodiments of the technology described herein. As shown in FIG. 10, implementation of the CRC coil significantly reduces the stray field, particularly at distances 1 meter or greater from the imaging region. Maximum reduction of the stray field occurs at a distance 4 meters from the imaging region. FIG. 11 illustrates another graph illustrating the stray field reduction when a CRC coil is used with an RF coil, in accordance with some embodiments of the technology described herein.

The technology described herein may be embodied in any of the following configurations:
(1) A radio frequency (RF) coil apparatus for facilitating magnetic resonance imaging (MRI) of at least a part of a patient's body that is positioned within an imaging region of an MRI system, the RF coil apparatus comprising: at least one primary RF coil configured to emit RF pulses and generate a first magnetic field during operation of the MRI system; and at least one secondary RF coil configured to generate a second magnetic field that, during operation of the MRI system, at least partially counteracts the first magnetic field in an external region outside of the imaging region of the MRI system.
(2) The RF coil apparatus of (1), wherein the first magnetic field extends over at least a portion of the imaging region and the external region outside of the imaging region.
(3) The RF coil apparatus of (2), wherein current in the at least one secondary RF coil is 180 degrees out of phase with current in the at least one primary RF coil.
(4) The RF coil apparatus of (3), wherein operation of the at least one secondary RF coil at least partially reduces a strength of an effective magnetic field in the external region outside of the imaging region of the MRI system.
(5) The RF coil apparatus of any one of (1) through (4), wherein the at least one primary RF coil and the at least one secondary RF coil are coupled to a common drive circuit, and wherein the at least one secondary RF coil comprises a plurality of counter rotating current loops connected in series with the at least one primary RF coil.
(6) The RF coil apparatus of any one of (1) through (5), wherein the at least one primary RF coil and the at least one secondary RF coil are electrically coupled.
(7) The RF coil apparatus of any one of (1) through (6), wherein the at least one primary RF coil and the at least one secondary RF coil are coupled to different drive circuits.
(8) The RF coil apparatus of any one of (1) through (7), wherein the at least one primary RF coil and the at least one secondary RF coil are not electrically coupled.
(9) The RF coil apparatus of any one of (1) through (8), wherein the at least one secondary RF coil consists of a single conductor.
(10) The RF coil apparatus of (9), wherein the single conductor is connected in series with the at least one primary RF coil.
(11) The RF coil apparatus of any one of (1) through (11), wherein the at least one secondary RF coil comprises multiple conductors.
(12) The RF coil apparatus of (11), wherein the multiple conductors are connected in series with the at least one primary RF coil.
(13) The RF coil apparatus of any one of (1) through (12), wherein the second magnetic field generated by the at least one secondary RF coil is directed substantially along a single axis.
(14) The RF coil apparatus of any one of (1) through (13), wherein the second magnetic field generated by the at least one secondary RF coil comprises a first component along a first axis and a second component along a second axis substantially perpendicular to the first axis.
(15) The RF coil apparatus of (14), wherein the at least one secondary RF coil is tilted relative to a third axis substantially perpendicular to the first and second axes.
(16) The RF coil apparatus of any one of (1) through (15), wherein the at least one secondary RF coil comprises multiple coils including a first coil and a second coil.
(17) The RF coil apparatus of (16), wherein the second magnetic field generated by the at least one secondary RF coil comprises a first component along a first axis generated by the first coil and a second component along a second axis substantially perpendicular to the first axis generated by the second coil.
(18) The RF coil apparatus of (16), wherein a current in the first coil is generated by a first power source and a current in the second coil is generated by a second power source different from the first power source.
(19) The RF coil apparatus of any one of (1) through (18), wherein the current in the at least one primary RF coil is generated by a first power source and a current in the at least one secondary RF coil is generated by a second power source different than the first power source.
(20) The RF coil apparatus of any one of (1) through (19), further comprising a frame comprising a first plate and a second plate disposed opposite the first plate, wherein the at least one primary RF coil is wound around the frame in a plurality of turns.
(21) The RF coil apparatus of (20), wherein the at least one primary RF coil comprises a plurality of conductors connected in series, the plurality of conductors being wound around the frame and forming the plurality of turns.
(22) The RF coil apparatus of (21), wherein the plurality of turns comprises at least 6 turns.
(23) The RF coil apparatus of any one of (1) through (22), wherein the RF coil apparatus is coupled to a base having a conveyance mechanism for transporting the RF coil apparatus to different locations.
(24) The RF coil apparatus of any one of (1) through (23), wherein the at least one secondary RF coil and the at least one primary RF coil comprise a same coil.
(25) The RF coil apparatus of any one of (1) through (24), wherein the at least one secondary RF coil and the at least one secondary RF coil comprise separate coils.
(26) A magnetic resonance imaging (MRI) system for imaging at least a part of a patient's body that is positioned within an imaging region of the MRI system, the MRI system comprising: a B₀ magnet that produces a B₀ magnetic field; and a radio frequency (RF) coil apparatus comprising: at least one primary RF coil configured to emit RF pulses and generate a first RF magnetic field during operation of the MRI system; and at least one secondary RF coil configured to generate a second RF magnetic field that, during operation of the MRI system, at least partially counteracts the first magnetic field in an external region outside of the imaging region of the MRI system.
(27) The MRI system of (26), further comprising an RF shield at least partially surrounding an imaging region of the MRI system, wherein the RF shield comprises at least one opening for receiving an anatomy of the patient.
(28) The MRI system of (27), wherein the at least one secondary RF coil is disposed inside the RF shield.
(29) The MRI system of (27), wherein the at least one secondary RF coil is disposed at least partially outside of the RF shield.
(30) The MRI system of (27), wherein the at least one secondary RF coil is disposed outside of the RF shield.
(31) The MRI system of (27), wherein the at least one secondary RF coil is disposed along the at least one opening of the RF shield.
(32) The MRI system of (27), wherein the at least one secondary RF coil comprises a loop having an area larger than an area of the at least one opening.
(33) The MRI system of any one of (26) through (32), wherein the B₀ magnetic field produced by the B₀ magnet is less than or equal to 0.2 T and greater than or equal to 0.1 T.
(34) The MRI system of any one of (26) through (33), wherein the B₀ magnetic field produced by the B₀ magnet has a strength less than or equal to 0.1 T and greater than or equal to 50 mT.
(35) The MRI system of any one of (26) through (34), wherein the first magnetic field extends over at least a portion of the imaging region and the external region outside of the imaging region.
(36) The MRI system of (35), wherein current in the at least one secondary RF coil is 180 degrees out of phase within current in the at least one primary RF coil.
(37) The MRI system of (36), wherein operation of the at least one secondary RF coil at least partially reduces a strength of an effective magnetic field in the external region outside of the imaging region of the MRI system.
(38) The MRI system of any one of (26) through (37), wherein the at least one primary RF coil and the at least one secondary RF coil are coupled to a common drive circuit, and wherein the at least one secondary RF coil comprises a plurality of counter rotating current loops connected in series with the at least one primary RF coil.
(39) The MRI system of any one of (26) through (38), wherein the at least one primary RF coil and the at least one secondary RF coil are electrically coupled.
(40) The MRI system of any one of (26) through (39), wherein the at least one primary RF coil and the at least one secondary RF coil are coupled to different drive circuits.
(41) The MRI system of any one of (26) through (40), wherein the at least one primary RF coil and the at least one secondary RF coil are not electrically coupled.
(42) The MRI system of any one of (26) through (41), wherein the at least one secondary RF coil consists of a single conductor.
(43) The MRI system of (42), wherein the single conductor is connected in series with the at least one primary RF coil.
(44) The MRI system of any one of (26) through (43), wherein the at least one secondary RF coil comprises multiple conductors.
(45) The MRI system of (44), wherein the multiple conductors are connected in series with the at least one primary RF coil.
(46) The MRI system of any one of (26) through (47), wherein the at least one secondary RF coil and the at least one primary RF coil comprise a same coil.
(47) The MRI system of any one of (26) through (48), wherein the at least one secondary RF coil and the at least one secondary RF coil comprise separate coils.
(48) A method of operating a radio frequency (RF) coil apparatus for facilitating magnetic resonance imaging (MRI) of at least a part of a patient's body that is positioned within an imaging region of an MRI system, the method comprising: driving at least one primary RF coil with a first current such that the primary RF coil emits RF pulses and generates a first magnetic field during operation of the MRI system; and driving at least one secondary RF coil with a second current such that the secondary RF coil generates a second magnetic field that at least partially counteracts the first magnetic field in an external region outside of the imaging region of the MRI system.
(49) The method of (48), wherein the second current is 180 degrees out of phase with the first current.
(50) The method of any one of (48) through (49), wherein the first magnetic field extends over at least a portion of the imaging region and the external region outside of the imaging region.
(51) The method of any one of (48) through (50), wherein the at least one primary RF coil and the at least one secondary RF coil are coupled to a common drive circuit, and wherein the at least one secondary RF coil comprises a plurality of counter rotating current loops connected in series with the at least one primary RF coil.
(52) The method of any one of (48) through (51), wherein the at least one primary RF coil and the at least one secondary RF coil are electrically coupled.
(53) The method of any one of (48) through (52), wherein the at least one primary RF coil and the at least one secondary RF coil are coupled to different drive circuits.
(54) The method of any one of (48) through (53), wherein the at least one primary RF coil and the at least one secondary RF coil are not electrically coupled.
(55) The method of any one of (48) through (54), wherein the at least one secondary RF coil consists of a single conductor.
(56) The method of (55), wherein the single conductor is connected in series with the at least one primary RF coil.
(57) The method of any one of (48) through (56), wherein the at least one secondary RF coil comprises multiple conductors.
(58) The method of (57), wherein the multiple conductors are connected in series with the at least one primary RF coil.
(59) The method of any one of (48) through (58), wherein the at least one secondary RF coil and the at least one primary RF coil comprise a same coil.
(60) The method of any one of (48) through (59), wherein the at least one secondary RF coil and the at least one secondary RF coil comprise separate coils.

### ALTERNATIVES AND SCOPE

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated. that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “substantially”, “approximately”, and “about” may be used to mean within ±20% of a target value in some embodiments, within ±10% of a target value in some embodiments, within ±5% of a target value in some embodiments, within ±2% of a target value in some embodiments. The terms “approximately” and “about” may include the target value.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A radio frequency (RF) coil apparatus for facilitating magnetic resonance imaging (MRI) of at least a part of a patient's body that is positioned within an imaging region of an MRI system, the RF coil apparatus comprising:
at least one primary RF coil configured to emit RF pulses and generate a first magnetic field during operation of the MRI system; and
at least one secondary RF coil configured to generate a second magnetic field that, during operation of the MRI system, at least partially counteracts the first magnetic field in an external region outside of the imaging region of the MRI system.

2. The RF coil apparatus of claim 1, wherein the first magnetic field extends over at least a portion of the imaging region and the external region outside of the imaging region.

3. The RF coil apparatus of claim 1, wherein current in the at least one secondary RF coil is 180 degrees out of phase with current in the at least one primary RF coil.

4. The RF coil apparatus of claim 1, wherein operation of the at least one secondary RF coil at least partially reduces a strength of an effective magnetic field in the external region outside of the imaging region of the MRI system.

5. The RF coil apparatus of claim 1, wherein the at least one primary RF coil and the at least one secondary RF coil are coupled to a common drive circuit, and wherein the at least one secondary RF coil comprises a plurality of counter rotating current loops connected in series with the at least one primary RF coil.

6. The RF coil apparatus of claim 1, wherein the at least one primary RF coil and the at least one secondary RF coil are electrically coupled.

7. The RF coil apparatus of claim 1, wherein the at least one primary RF coil and the at least one secondary RF coil are coupled to different drive circuits.

8. The RF coil apparatus of claim 1, wherein the at least one primary RF coil and the at least one secondary RF coil are not electrically coupled.

9. The RF coil apparatus of claim 1, wherein the at least one secondary RF coil consists of a single conductor.

10. The RF coil apparatus of claim 9, wherein the single conductor is connected in series with the at least one primary RF coil.

11. The RF coil apparatus of claim 1, wherein the at least one secondary RF coil comprises multiple conductors.

12. The RF coil apparatus of claim 11, wherein the multiple conductors are connected in series with the at least one primary RF coil.

13. The RF coil apparatus of claim 1, wherein the second magnetic field generated by the at least one secondary RF coil is directed substantially along a single axis.

14. The RF coil apparatus of claim 1, wherein the second magnetic field generated by the at least one secondary RF coil comprises a first component along a first axis and a second component along a second axis substantially perpendicular to the first axis.

15. The RF coil apparatus of claim 14, wherein the at least one secondary RF coil is tilted relative to a third axis substantially perpendicular to the first and second axes.

16. A method of operating a radio frequency (RF) coil apparatus to facilitate magnetic resonance imaging (MRI) of at least a part of a patient's body that is positioned within an imaging region of an MRI system, the method comprising:
emitting, using at least one primary RF coil of the RF coil apparatus, RF pulses to generate a first magnetic field during operation of the MRI system; and
generating, using at least one secondary RF coil, a second magnetic field that, during operation of the MRI system, at least partially counteracts the first magnetic field in an external region outside of the imaging region of the MRI system.

17. The method of claim 16, wherein the first magnetic field extends over at least a portion of the imaging region and the external region outside of the imaging region.

18. The method of claim 16, wherein current in the at least one secondary RF coil is 180 degrees out of phase with current in the at least one primary RF coil.

19. The method of claim 16, wherein the at least one secondary RF coil is operated to at least partially reduce a strength of an effective magnetic field in the external region outside of the imaging region of the MRI system.

20. The method of claim 16, wherein the second magnetic field generated by the at least one secondary RF coil is directed substantially along a single axis.
